(19) United States
(12) Patent Application Publication
(10) Pub. No.: US 2021/0130769 A1
(43) Pub. Date: May 6, 2021

(54) Method for Single Cell Encapsulation via Metabolic Glycoengineering and Copper-Free Click Chemistry

(71) Applicant: The Board of Trustees Of the Leland Stanford Junior University, Stanford, CA (US)

(72) Inventors: Byeongtaek Oh, Columbia, MD (US); Sruthi Santhanam, Menlo Park, CA (US); Paul George, Sunnyvale, CA (US)

(21) Appl. No.: 17/090,064

(22) Filed: Nov. 5, 2020

Related U.S. Application Data
(60) Provisional application No. 62/931,518, filed on Nov. 6, 2019.

(51) Int. Cl. C12N 5/00

(52) U.S. Cl. CPC C12N 5/0012; C12N 5/0006

(57) ABSTRACT

A method of single-cell encapsulation of cells using glycoengineering and click-chemistry is provided. Cells are treated with a precursor for metabolic engineering to modify glycans in a cell membrane and form reactive component A-glycans in the cell membrane suitable for a click-chemistry reaction. The treated cells are suspended in a polymer solution which has a reactive component B suitable for the click-chemistry reaction. The reactive component A-glycans react via the click-chemistry with the reaction component B thereby forming single cell polymer encapsulated cells. Applications include optimizing stem cell function, cell to cell crosslinking, formation of networks of cells or organoids, functionalizing the cells with reactive groups or attaching the cells to a substrate or surface.

### CROSS-REFERENCE TO RELATED APPLICATIONS

This application claims priority from U.S. Provisional Patent Application 62/931,518 filed Nov. 6, 2019, which is incorporated herein by reference.

### STATEMENT OF GOVERNMENT SPONSORED SUPPORT

This invention was made with Government support under contract NS089976 awarded by the National Institutes of Health. The Government has certain rights in the invention.

### FIELD OF THE INVENTION

This invention relates to polymer-mediated cell encapsulation methods.

### BACKGROUND OF THE INVENTION

Stem cell therapy is a promising treatment option for patients with cardiac, orthopedic, and neurological diseases. Several clinical trials have demonstrated the successful and safe delivery of stem cells in the target region and their ability to improve functional outcomes. However, there is still a need to improve the therapeutic efficacy, especially for neural deficits, and to prolong cell survival in the often harsh target environment. Moreover, often hundreds of millions of stem cells are transplanted to obtain a beneficial therapeutic effect, thereby increasing the time, cost, and risk associated with these procedures. To overcome the limitations, polymer-mediated cell encapsulation methods have been applied to therapies aimed at restoring injured tissue, particularly in central nervous system disorders.

Biomaterials have been extensively explored to deliver neural stem cells and to promote robust cell growth, survival, and trophic factor release. This has led to a myriad of developments, such as programmable and injectable hydrogels serving as cell scaffolds. To synthesize these commonly employed vehicles, building blocks such as hyaluronic acid or chitosan are chemically or physically cross-linked often with azide-alkyne bonds or ionic interactions. These biomaterials and methods for encapsulation have yielded results of varying degrees of success, ranging from preclinical development to advanced clinical trials. However, a limitation in current methods of cell encapsulation lies in the fact that there is often a plateau in regards to the maximal therapeutic effect derived from a single transplant. Additionally, simultaneous encapsulation of a group of neural stem cells in a hydrogel depot commonly leads to uneven coverage of these cells, creating inconsistent and varying extracellular matrix (ECM) environments for each individual cell. A uniform method of cellular encapsulation could produce more reliable and effective results, and the present invention addresses this need. The invention also addresses the ability to individually coat stem cells which provides the opportunity to direct stem cell interactions with other cells or surfaces.

### SUMMARY OF THE INVENTION

The present invention emphasizes a copper-free click-chemistry powered three-dimensional, single-cell encapsulation method aimed at synthesizing an optimal polymer coating around each cell, in particular, neural stem cells. In accordance with the present disclosure, the polymer encapsulation of neural stem cells markedly increased the release of neurotrophic factors, and hence, the therapeutic efficacy of the cells. The invention also emphasizes the use of the method for enhancing cellular trophic factor release, cellular/neural network re-construction, single-cell control, and organoids development.

The present invention is a method for a uniform single-cell encapsulation using polymers to enable precise control of cellular interactions with the extracellular matrix (ECM). The invention emphasizes its use for enhancing cellular trophic factor release, cellular/neural network re-construction, single-cell control and organoids development.

Polymer-mediated cell encapsulation methods have been explored to deliver stem cells and to promote robust cell growth, survival, and trophic factor release. The critical step is to develop a method of cellular encapsulation that could increase the therapeutic efficacy of the encapsulated cells. However, the limitation is in the simultaneous encapsulation of a group of cells in a hydrogel depot that commonly leads to uneven coverage of these cells, creating inconsistent and varying interactions of each cell with its extracellular matrix environments.

To resolve this problem, a single-cell encapsulation method via click-chemistry and glycoengineering technique was developed. This technique creates an efficient way to coat a layer of polymer around each cell. By varying the stiffness of the polymer coating, the proteins released by the cells were modulated. The optimized tactile interactions with the polymeric ECM enhance trophic factor release. By augmenting the therapeutic benefit of each cell, the number of cells needed to cause a therapeutic effect in a biological system can be reduced. It is, therefore, an objective of the present invention to increase the therapeutic efficacy of each cell. In addition, because each individual cell is encapsulated, further modification of the polymer can be used to direct cellular attachments to other cells or surfaces.

The single-cell encapsulation method presented in this invention expand their utility beyond the conventional encapsulation of a group of cells in a polymeric hydrogel depot. The technique with the ability to manipulate the cellular interaction with the extracellular environment can act as a fundamental regulator of cell function. For example, single-cell encapsulation of neural stem cells markedly increased the release of neurotrophic factors such as VEGF and CNTF. Given the promise of stem cell therapeutics, the present invention to uniformly enhance stem cell function could prove transformative in improving efficacy and increasing feasibility by reducing the total number of cells required. Moreover, the individual cellular control achieved via the present invention expands the ability of the method to reconstruct cellular networks, in particular, neural cells network, and in the development of the organoids. Because each cell is individually coated, the polymer can be modified (1) to allow for combinations of different cells with different polymers to form engineered combinations and (2) to attach moieties to each cell that could guide it to a particular binding target for single cell manipulations.

Significant advantages of this technique are provided. Stem cell therapy is a promising treatment option for patients with cardiac, orthopedic, and neurological diseases. Several clinical trials have demonstrated the successful and safe delivery of stem cells in the target region and their ability to improve functional outcomes. However, there is still a need to improve the therapeutic efficacy, especially for neural deficits such as stroke, Alzheimer's and Parkinson's, and to prolong cell survival in the often harsh target environment. Moreover, a very high dosage of stem cells is often delivered to obtain a beneficial therapeutic effect, thereby increasing the time, cost, and risk associated with these procedures.

With the use of single-cell encapsulation, the therapeutic efficacy of the transplanted cells can be improved. Because the therapeutic benefit of each cell can be augmented, the number of cells needed for transplantation can be reduced. Moreover, the encapsulation technique creates the opportunity to better understand the activity of stem cells at a cellular level, which is essential to designing effective cellular modulation strategies and translational therapeutics. Thus, the single-cell encapsulation technique could emerge as a translatable, non-viral cell modulation method and has the potential to improve stem cells' therapeutic effect.

In one aspect, the invention can be described as a single-cell encapsulation of cells via click chemistry having the following steps:
1. Cells from their original plate or flask were lifted and re-plated at a high density on a 6-well plate.
2. Cells were then incubated under standard cell culture conditions (37° C., 5% CO₂) for 24 hr.
3. The cells at 80% confluency were treated with N-azidoacetylmannosamine-tetraacylated (Ac₄ManNAz; 10 μM for neural progenitor cells (NPC)) (Kerafast, Boston, Mass.) for 2 days. More generally speaking the cells can be treated with a precursor for metabolic engineering. Examples of such a precursor are:
   Tetraacetylated N-azidoacetyl-D-mannosamine,
   Tetraacetylated N-azidoacetyl-D-galactosamine, and
   Tetraacetylated N-azidoacetyl-D-glucosamine.
   For any other molecule or cell type, the concentration of the azide containing molecule should be optimized. In this example, 10 μM of Ac₄ManNAz for the NPCs.
4. The cells were washed with PBS and trypsinized from the plates with Accutase or appropriate trypsinization solution.
5. The cells were collected by centrifugation and re-suspended in the media containing different molecular weights of dibenzocyclooctyne-polyethyl glycol (DBCO-PEG; For the experiments, NPCs were re-suspended in DBCO-PEG of 5, 10, 20, and 30 kDa; 100,000 cells/mL) (BroadPharm, San Diego, Calif.) at a concentration of 1 μg/mL for 1 hr at 37° C.
6. Subsequently, the cells were rinsed with PBS and re-suspended in the maintenance media.

Click chemistry refers to a group of reactions that are fast, simple to use, easy to purify, versatile and give high product yields. There are a series of reactions which are classified into 4 main types—cycloadditions, Nucleophilic ring-openings, Carbonyl chemistry of the non-aldol type, Additions to carbon-carbon multiple bonds (Please refer to Hein et al. Click Chemistry, a Powerful Tool for Pharmaceutical Sciences, Pharm Res 2008: 25(1) 2216-2230). For the purposes of this invention, a copper-free cycloaddition of azide (Ac₄ManNAz) to alkyne (DBCO-PEG, DBCO has the alkyne group) was used. However, one could use any of the other click reactions for single-cell encapsulation.

Copper-free refers to the reaction between Ac₄ManNAz and DBCO-PEG which occurs in a medium without copper.

Trypsinization is the process of cell dissociation using trypsin, a proteolytic enzyme which breaks down proteins, to dissociate adherent cells from the flasks/plate in which they are being cultured.

The present invention is also defined as a method of single-cell encapsulation of cells using glycoengineering and click-chemistry, which in one example is a copper-free method. In this method, cells are treating with a precursor for metabolic engineering to modify glycans in a cell membrane. The treatments forms reactive component A-glycans in the cell membrane suitable for a click-chemistry reaction. Examples of the precursor are Tetraacetylated N-azidoacetyl-D-mannosamine, Tetraacetylated N-azidoacetyl-D-galactosamine, or Tetraacetylated N-azidoacetyl-D-glucosamine. Further to the method, the treated cells are suspended in a polymer solution. The polymer solution has a reactive component B suitable for the click-chemistry reaction. Herewith, the reactive component A-glycans in the cell membrane reacts via the click-chemistry with the reaction component B thereby forming single cell polymer encapsulated cells. Examples of the polymer solution are dibenzocyclooctyne-polyethyl glycol (DBCO-PEG), DBCO-PEG-NH₂, DBCO-PEG-NHS Ester, DBCO-PEG-COOH, 4-arm-PEG-DBCO, or DBCO-PEG-DBCO.

In one variation to the method, the polymer solution has polymers with different molecular weights, and the cells in the formed single cell polymer encapsulated cells each have a different polymer molecular weight. In one example, molecular weights can range from 5 to 75 kDa.

In another variation to the method, the single cell polymer encapsulated cells can be crosslinked to each other.

In yet another variation to the method, a network of cells or organoids can be formed by crosslinking the single cell polymer encapsulated cells to each other.

In yet another variation to the method, the single cell polymer encapsulated cells can be functionalized with a reactive functionalized group.

In still another variation to the method, the single cell polymer encapsulated cells can be attached or adhered to a substrate or surface.

### BRIEF DESCRIPTION OF THE DRAWINGS

FIG. 1 shows according to an exemplary embodiment of the invention a schematic illustration of the single-cell encapsulation of NPCs via click-chemistry. 1) NPCs are treated with Ac4ManNAz to produce azide modified cells. 2) NPCs expressing glycan protein modified with azide (—NH3) are encapsulated with varying molecular weights of PEG (R) via a click crosslinking reaction between the azide group and DBCO modified PEGs with the alkyne group.

FIGS. 2A-D show according to an exemplary embodiment of the invention optimization of cell encapsulation parameters. FIGS. 2A-B: Optimization of the concentration and time parameters for effective homogenous and individual coating of cells with the PEG polymer. Maximization of the fluorescent intensity (F.I.) seen due to the FL545 moiety of the coated PEG polymer corroborates this optimization. FIG. 2C: Live/dead images demonstrate high viability of encapsulated cells. FIG. 2D: IFC images illustrate the relationship between the various concentrations of the DBCO-PEG-FL545 and the resulting illumination. Control is the cells without encapsulation (+Ac₄ManNAz, 0 ng/mL polymer).

FIGS. 3A-B show according to an exemplary embodiment of the invention single cell encapsulation. FIG. 3A: TEM image of a PEG (30 kDA) coated cell. The arrows might trace a layer of polymer along the cell membrane in the intercellular space (*) between two cells. FIG. 3B: IFC image suggests encapsulation of the cell with PEG-FL545 coating (right) compared to the control (left). The FL545 moieties contribute to the red fluorescence around the nucleus stained with DAPI (blue fluorescence 310).

FIGS. 4A-C show according to an exemplary embodiment of the invention different molecular weights of polymer encapsulation affect trophic factor release. FIG. 4A: Bar graphs portraying the trend of increasing transcription of neurotrophic factor release (measured using qRT-PCR) in relation to the various weights of the polymer chain used for encapsulation. The factor expression corresponding to each of the different polymer weights used is denoted in terms of fold change (F.C.) with respect to the control group without encapsulation. FIG. 4B: Pearson's coefficient analysis is also provided to denote the correlation between proper encapsulation and increased factor release. FIG. 4C: ELISA results indicating increased VEGFB production with various molecular weights of DBCO-PEG. FIG. 4B and FIG. 4C: Analyzed using a one-way ANOVA, followed by Tukey's HSD post hoc test with **P<0.01. In FIG. 4B, VEGFB was statistically significant compared to other groups. In FIG. 4C, 30 kDa polymer encapsulation was statistically different from other groups. Values represent the mean of independent experiments (n=4); error bars, S.D.

FIG. 5 shows according to an exemplary embodiment of the invention higher molecular weight PEG produces a softer polymeric ECM. The AFM analysis illustrates that the encapsulation method modulates the Young's Modulus of the ECM surrounding the cells. Analyzed using a one-way ANOVA, followed by Tukey's HSD post hoc test with **P<0.01. 30 kDa group was statistically significant compared to other groups. Values represent the mean of independent experiments (n=4); error bars, S.D.

FIGS. 6A-B show according to an exemplary embodiment of the invention a softer layer of polymer encapsulation surrounding the cells augments ADCY8 expression and cAMP production. FIG. 6A: qPCR-RT results demonstrate the up-regulation of ADCY8 with softer (higher molecular weight) PEG. FIG. 6B: Bioluminescent results show an increase in cAMP with softer PEG. FIGS. 6A-B: Analyzed using a one-way ANOVA, followed by Tukey's HSD post hoc test with **P<0.01. 30 kDa group was statistically significant compared to other groups. Values represent the mean of independent experiments (n=4); error bars, S.D.

FIGS. 7A-B show according to an exemplary embodiment of the invention inhibition and activation of actin polymerization alters trophic factor release. This trend is shown in cells encapsulated by both the 5 kDa and 30 kDa polymers. FIG. 7A: Using cells encapsulated with 5 kDa PEG, VEGFB levels with CytoD treatment was significantly different from other groups with ELISA analysis FIG. 7B: Using cells encapsulated with 30 kDa PEG, VEGFB levels with LPA treatment was significantly different from other groups with ELISA analysis. In FIGS. 7A-B: Analyzed using a one-way ANOVA, followed by Tukey's HSD post hoc test with **P<0.01. Values represent the mean of independent experiments (n=4); error bars, S.D.

FIG. 8 shows according to an exemplary embodiment of the invention a graphical illustration of the interplay between click-chemistry DBCO-PEG single cell encapsulation system and actin polymerization, stressing how it affects the ACDY-cAMP transduction pathway and factor release.

FIG. 9 shows according to an exemplary embodiment of the invention cell to cell crosslinking. Immunofluorescent image of NPCs coated with DBCO-PEG5-NH2 (green fluorescence 910) and those coated with DBCO-PEG5-NH2 ester (red fluorescence 920), which interact forming an amid bond.

FIGS. 10A-B show according to an exemplary embodiment of the invention microfabrication techniques to pattern encapsulated single cells. FIG. 10A shows a schematic of single cell encapsulated cells attached to patterned gold substrate 1010. FIG. 10B shows a schematic of two cell types attached to a patterned gold 1010 (blue cells 1020) and silicon nitride 1030 (orange cells 1040).

### DETAILED DESCRIPTION

Glycoengineering is a technique that allows manipulation of cellular membrane glycans, and is an intriguing method to homogenously regulate paracrine properties at a cellular level. In this invention, a single-cell encapsulation method via click-chemistry and glycoengineering is provided. This technique creates an efficient way to coat a layer of polymer around each neural progenitor cell (NPC).

By varying the stiffness of the polymer coating, one would be able to modulate the proteins released by the cells. The optimized tactile interactions with the polymeric coating around cell enhance trophic factor release, such as VEGF. By augmenting the therapeutic benefit of each NPC, the number of cells needed to cause a therapeutic effect in a biological system can be reduced.

### Click-Chemistry Powered Glycoengineering

The stiffness of the ECM can vary greatly with the extremes seen in pathologic conditions such as cancer and glioma. The stiffness of the surrounding cell environment, as defined by this relationship between stress and strain using Young's Modulus, plays a critical role in dictating cellular function, proliferation, and survival.

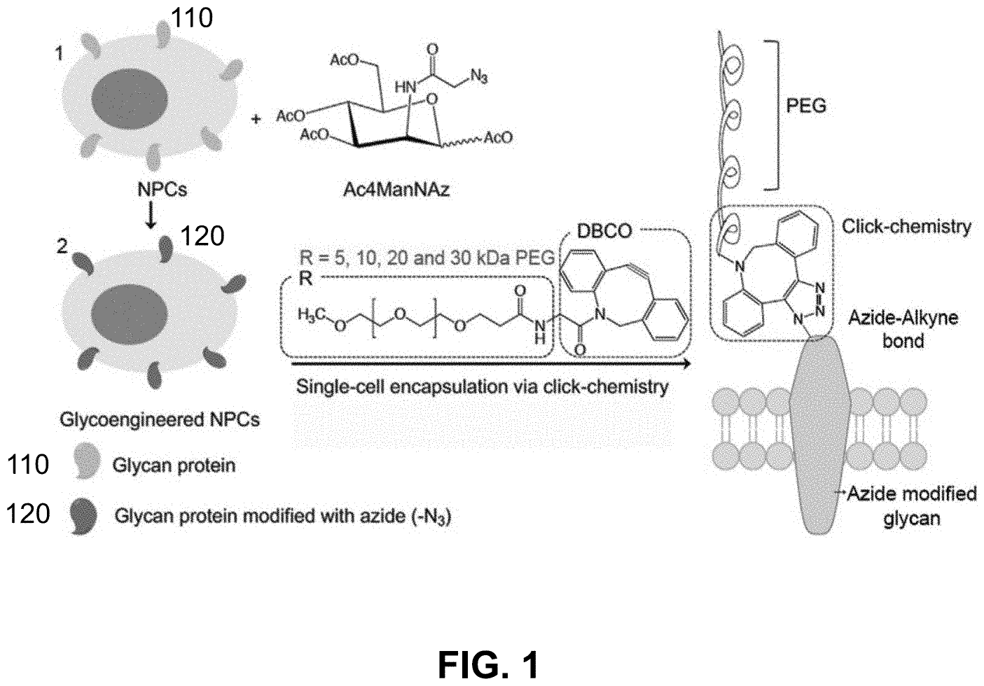

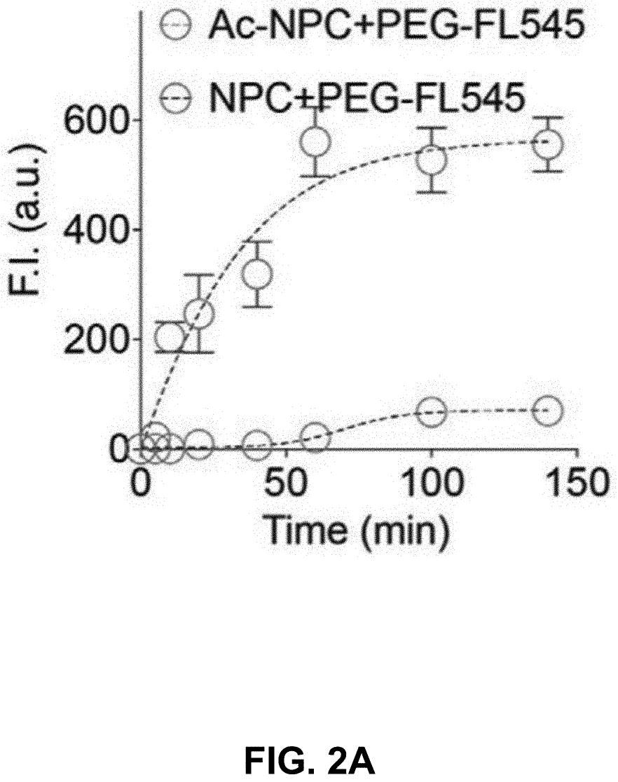

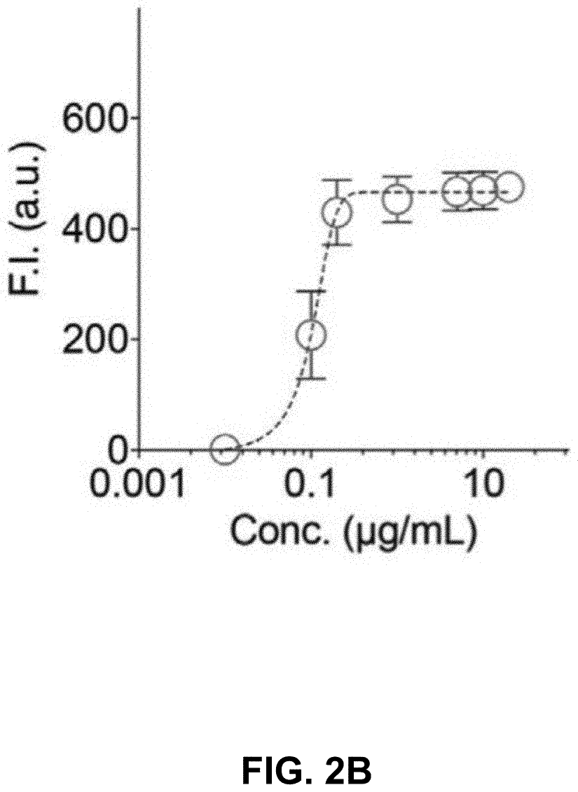

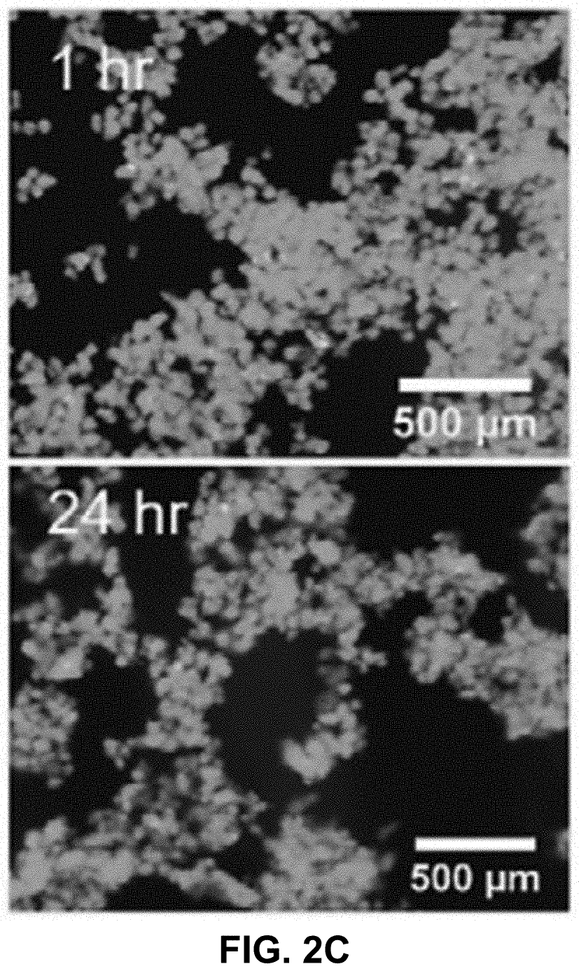

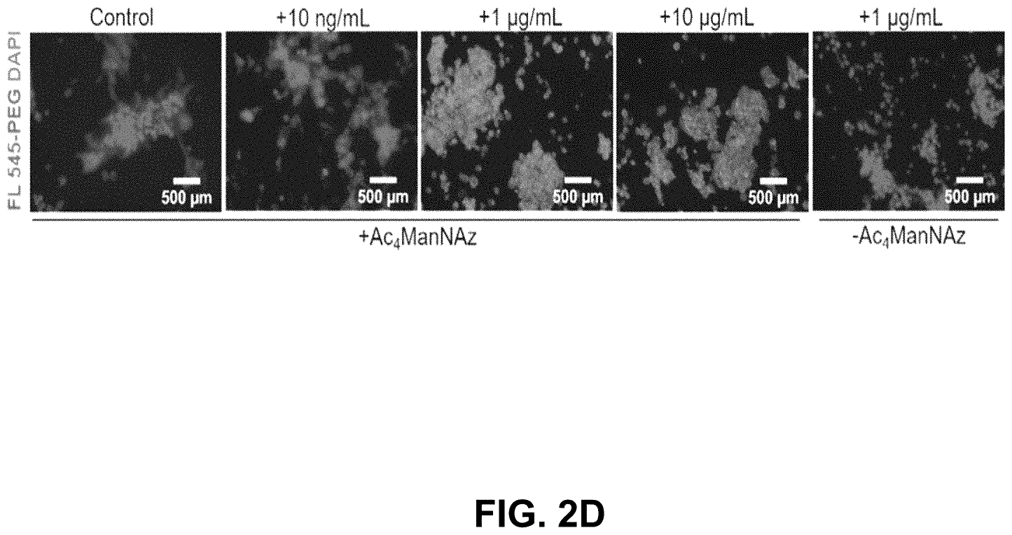

To determine the optimal stiffness of the polymer coating for the single cell encapsulation technique, the inventors evaluated DBCO-PEG chain coatings of various molecular weights (5, 10, 20, and 30 kDa) attached via click-chemistry (FIG. 1). By varying the molecular weight of the polymer attached to the NPCs, the stiffness of the immediate environment surrounding the cells could be altered, resulting in cellular control of differing capabilities and properties. First, the required incubation time of cells was examined incorporated with the Ac₄ManNAz attachment moiety in media containing a FL545-tagged DBCO-PEG polymer. Subsequently, the ideal concentration of the DBCO-PEG for maximal cellular encapsulation was evaluated. Based on the fluorescence intensity readings, the inventors found that attachment of the polymer reached a maximal plateau beginning at approximately 60 min of incubation (FIG. 2A) and 1 μg/mL of PEG in the media (FIG. 2B). Thus, these parameters were utilized for the remainder of the experiments. The viability of cells encapsulated with varying molecular weight of PEG were determined and cells encapsulated with 30 kDa PEG experienced minimal cell death after the incubation for 1 hr and 24 hr (FIG. 2C). Further, in one of the control group, NPCs without the glycans modified with Ac₄ManNAz, a strong signal of FL 545-PEG was not observed in the fluorescent microscopy images of cells compared to those coated with different concentrations of FL 545-PEG (FIG. 2D). This indicates the cells were only encapsulated by polymer if the Ac₄ManNAz moiety was present. Interestingly, while growing in culture, the encapsulated cells appear to aggregate in a similar pattern as unencapsulated cells, in effect forming a multi-cellular nano-encapsulation. If cells are encapsulated individually through the click chemistry method even if some level of aggregation occurs at higher densities, single cell control would still be maintained by the immediate cellular environment provided by the single cell polymeric coatings.

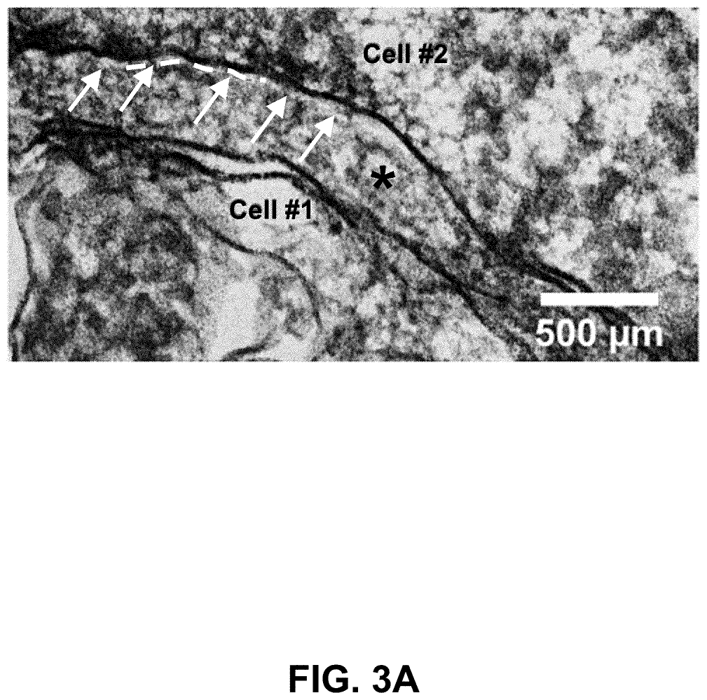

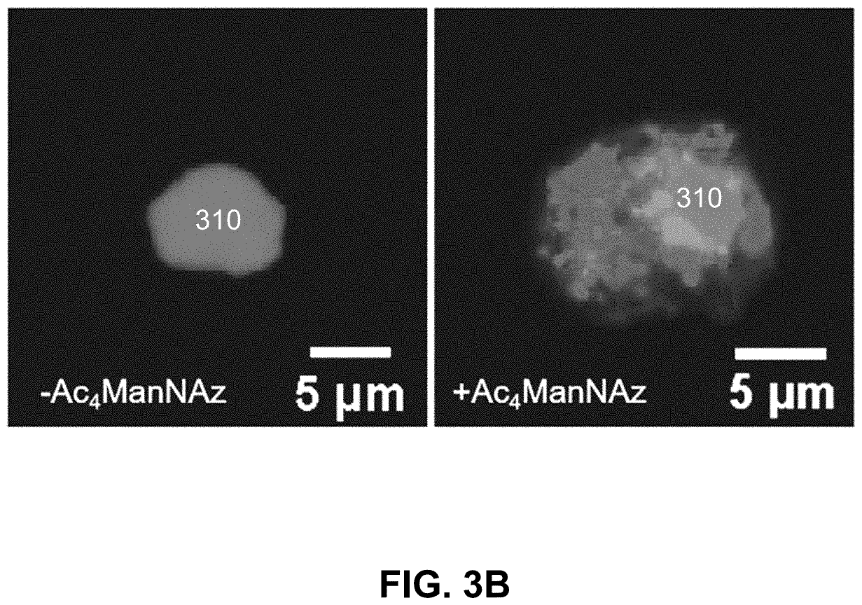

To ascertain that individual cells were indeed being coated, the cells were visualized with fluorescent microscopy (BZ-X710, Keyence, Itasca, Ill.) and transmission electron microscopy (TEM, JEM-1400, JEOL solutions, Peabody, Mass.). High magnification images of fluorescently-tagged PEG were obtained to verify a layer of polymer surrounding individual cells. The images reveal a layer of red fluorescence around the NPC with the Ac₄ManNAz moiety, confirming a single-cell nano-encapsulation with the FL 545-PEG (FIG. 3B). A TEM image of a 30 kDa PEG coated NPC sample show a layer of different grayscale along the cell membrane (FIG. 3A), which may suggest a layer of polymer coating around the cell.

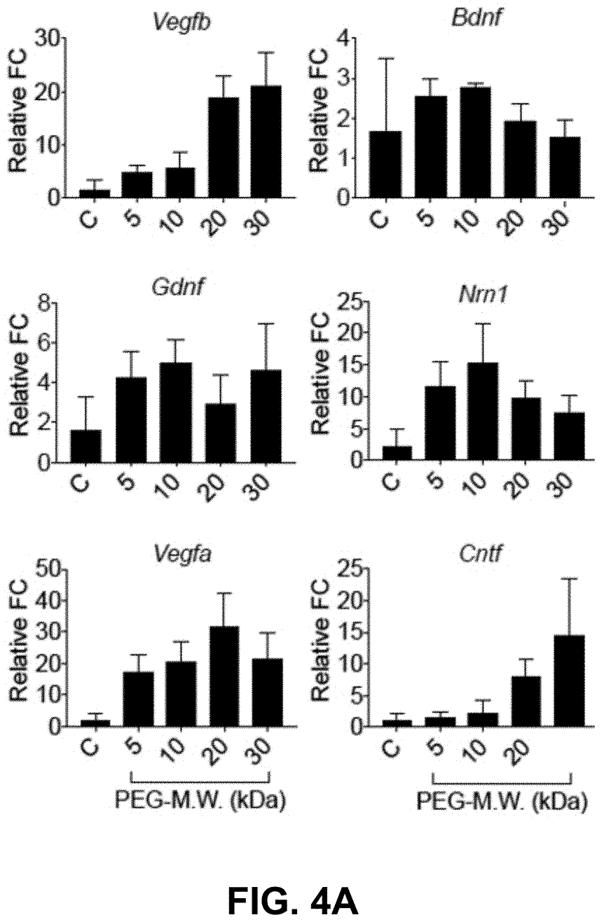

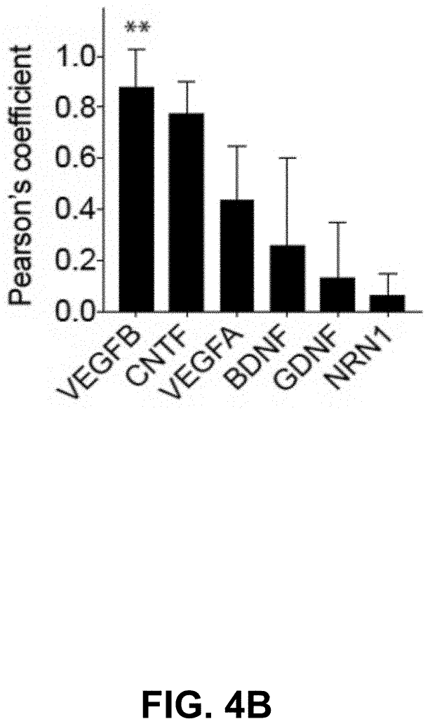

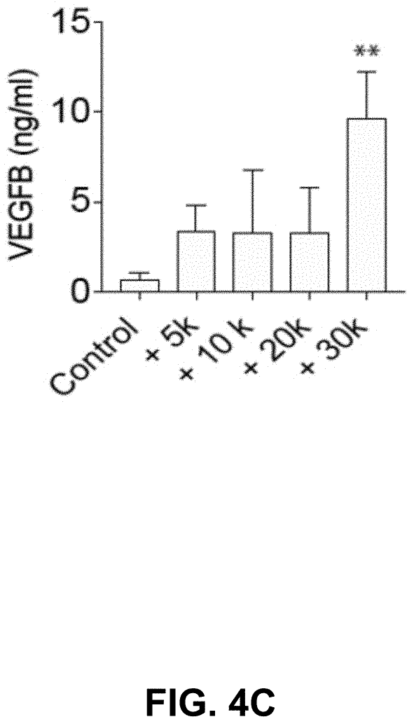

Verification of NPC Modification by Encapsulation Polymers have been shown to modulate the inherent mechano-sensing properties of cells. To evaluate if the polymer modified the cellular properties of the NPCs, the transcription of trophic factor released by the polymer-encapsulated NPCs were evaluated using qRT-PCR. It was observed that polymer encapsulation caused an increase in trophic factor release (FIG. 4A) compared to the control (C), which are cells without encapsulation. The augmentation of factor release is clearly exemplified based on the fold increases in the release of various important neurotrophic molecules such as VEGFA, VEGFB, BDNF, CNTF, GDNF, and NRN1. For many factors, the coatings of higher molecular weight result in higher fold factor release. Both VEGFA and VEGFB showing the largest increases at almost 20-fold change compared to the unencapsulated control group (FIG. 4A). To confirm that varying the molecular weight of the polymer was indeed the variable accounting for the increased trophic factor release, Pearson's coefficients for different trophic factors were plotted based on the qRT-PCR analyses. The Pearson's coefficients, a measure of linear correlation between trophic factor release and polymer encapsulation, confirm the effectiveness of our methods. All of the factors have a correlation greater than 0, with VEGFB having the strongest positive correlation with a coefficient of almost 0.88±0.15 (FIG. 4B). The factors with the highest Pearson's coefficients align with previous studies that show VEGF and CNTF respond to mechanical stretch. Notably, members of the neurotrophin family, BDNF and NRN1, had smaller Pearson's coefficients, indicating this family of proteins may be less responsive to mechanical stimuli.

An ELISA study was conducted to measure the concentration of VEGFB in the supernatant to determine if the gene modifications resulted in a change in protein concentration of VEGFB (the factor with the highest Pearson coefficient). The concentration of the factor released in the media isolated from the encapsulated cells is almost a factor of 10 greater in the 30 kDa group (9.7±2.5 ng/mL) compared to the control group (0.7±0.3 ng/mL) (FIG. 4C, P<0.01). This further corroborates the fact that the technique effectively modulates factor release from NPCs.

### Analysis of Changes in Tropic Factor Release Due to Manipulation of Mechanical Cues

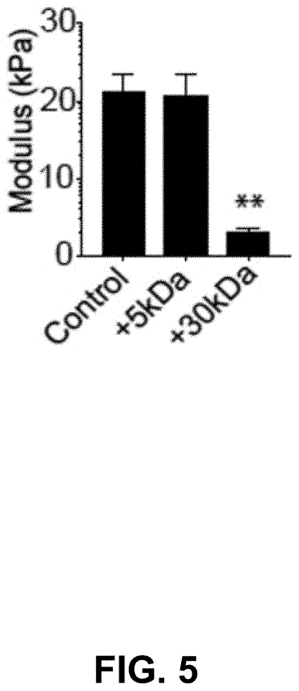

To further investigate whether the increase in trophic factor production is associated with polymer mechanical characteristics, atomic force microscopy (AFM) was used to determine the stiffness of an individual NPC's surface modified with polymer. Based on the results from neurotrophic factor release, the experimental groups that were best representatives, including a control group without polymer, and NPCs modified with 5 kDa and 30 kDa PEG were chosen for this study. The Young's elastic modulus is a measure of a substance's ability to resist deformation. It is calculated by dividing the stress placed on the substance in question by the strain it experiences. The AFM technique was able to measure the Young's modulus of individual NPCs from the control, 5 kDa PEG, and 30 kDa PEG groups. Because the control group consists of cells alone, the Young's modulus is the measurement of stiffness from the cellular surface, which is primarily produced by the cytoskeleton, nucleus and other internal organelles. The neural progenitor cell stiffness was found to be about 20 kPa (FIG. 5). Interestingly, the 5 kDa PEG-coating had a similar stiffness to the cells alone (the control group). However, cell encapsulation with polymers of a larger molecular weight (30 kDa PEG) exhibit a smaller Young's modulus, indicating an environment that is softer than that of the 5 kDa PEG (FIG. 5). Compared to the 5 kDa polymer groups, individually encapsulating NPCs with a polymer weighing 30 kDa resulted in an ECM with a stiffness lowered by almost a factor of 10 based on the Young's moduli measurements (FIG. 5). These results are consistent with previous findings that the stiffness of PEG decreases with higher molecular weights. Presumably, this is related to the fact that there is a change in crosslink density of PEG with varying molecular weight, thereby producing less stiff polymers with an increase in its molecular weight In addition, The uniformity of each single cell that was probed indicates single cell nano-encapsulation results from this technique in these conditions. These results elucidate the ability of the cell encapsulation technique to modulate the NPCs' immediate surrounding environment.

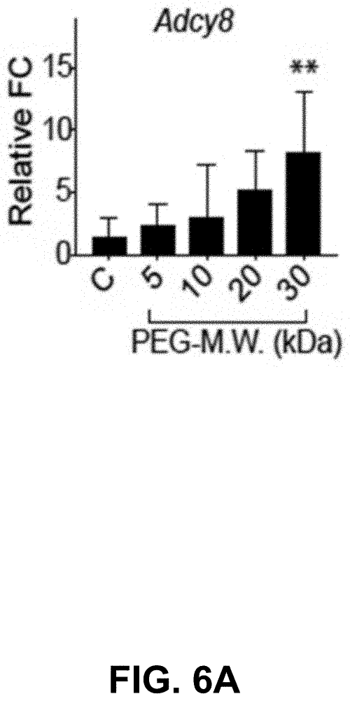

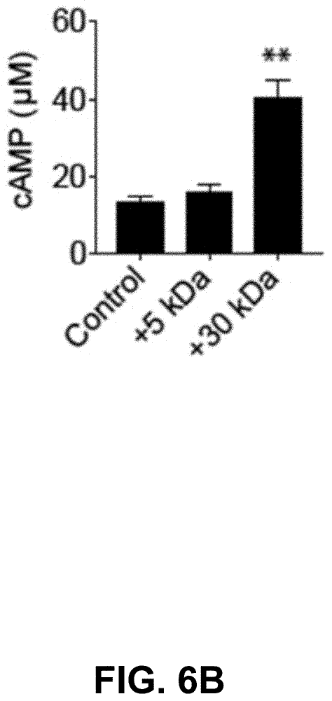

To delineate which pathways may play a role in converting the mechanical signals into increased trophic factor release, an important pathway in cell signaling was studied; the cyclic adenosine monophosphate (cAMP) dependent pathway. It has been demonstrated that cAMP signaling activated by mechanical stimuli is produced at the cell surface. cAMP is also known to regulate cell paracrine factor expression. External mechanical cues can activate adenylyl cyclase, which catalyzes conversion of ATP to cAMP. Specifically, adenylate cyclase 8 (ADCY8) plays an important role in cAMP regulation. Thus, the inventors analyzed ADCY8 and cAMP levels using qRT-PCR and a luminometric assay (FIGS. 6A-B, respectively) to determine if they were altered by the properties of the PEG. Cells coated with softer PEG (30 kDa) upregulated ADCY8 as compared to the other groups. Additionally, cAMP conversion was very efficient in cells coated with softer PEG. These results suggest that cell encapsulation with the soft polymer alters mechanical stress-induced cAMP signaling, resulting in the increased production of trophic factors. These findings support prior results demonstrating the upregulation of ADCY8 leads to higher production of cAMP.

### Inhibition Experiments to Confirm Variation of ADCY8-cAMP Mechanism During Mechanical Stimulation

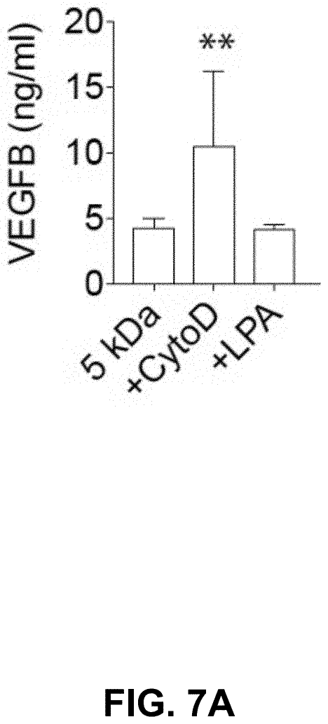

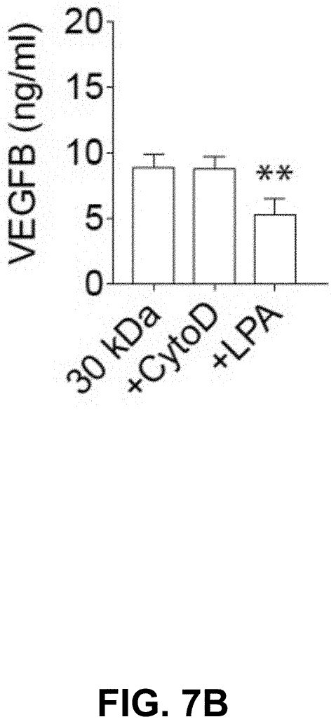

Increasing the levels of cAMP in a cell lowers the levels of actin polymerization. One of the primary methods that a cell reacts to an increased stiffness of the ECM involves the actin cytoskeleton through actin dynamics. Because of actin's role in the cAMP pathway and mechanotransduction, the inventors investigated the effect of inhibitors and activators of actin dynamics in response to the mechanical stimuli of PEG. Since VEGFB had been the trophic factor most largely effected by the mechanical properties of the coated polymer, we explored the role of actin and its effect on VEGFB release (FIGS. 7A-B).

CytoD inhibits actin polymerization. Theoretically, if the cAMP pathway was increased as seen in the soft PEG condition, CytoD would have less effect on trophic factor release (i.e. VEGFB) because the actin pathway would already be inhibited by cAMP upregulation. Indeed in the experiments, the inventors found that CytoD increased VEGFB production in NPCs coated with 5 kDa PEG (FIG. 7A). However, in NPCs coated with 30 kDa PEG, the VEGFB concentration was not significantly different, indicating that actin polymerization was already sufficiently inhibited by the softer polymer encapsulation.

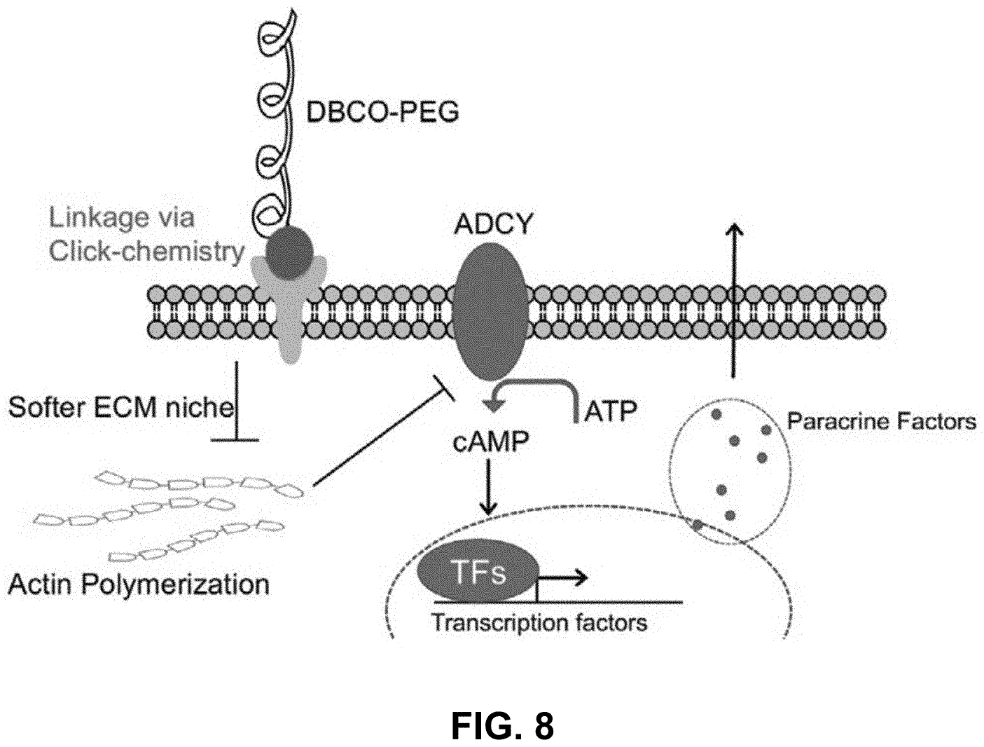

Given these results it was hypothesized that an activator of actin would have the opposite effect. To further demonstrate this, lysophosphatidic acid (LPA, an activator of actin polymerization) was applied to the encapsulated NPCs. Because the 30 kDa encapsulated NPCs inhibited actin polymerization, the reversal of actin inhibition through LPA results in decreased production of VEGFB (FIG. 7B). Taken together, these results indicate that actin polymerization plays an important role in extracellular polymer regulated trophic factor release in the encapsulated cells (FIG. 8).

### CONCLUSION

Polymeric cell encapsulation is an effective method to increase the survival and efficacy of cell transplantation. The development of a uniform nano-encapsulation technique described above allows for precise control of cellular trophic factor release by leveraging a cell's response to its extracellular polymer coating. In addition to optimizing cellular function, the use of glycoengineering to form a consistent cellular encapsulation technique creates the opportunity to better understand the activity of stem cells at a cellular level. This understanding is essential to designing effective cellular modulation strategies and translational therapeutics. Further modification of the polymer coating using this methodology could also be used to direct cellular attachments to other cells or surfaces, thereby paving a way for cellular/neural network reconstruction.

To conclude, by applying the single-cell encapsulation technique via click-chemistry, the inventors were able to investigate the effect of single cell encapsulation on trophic factor release. The inventors discovered a feasible mechanism by which the molecular weight of the polymer controls cell surface stiffness and regulates cell signaling via modulation of the ADCY8-cAMP pathway. Changes in ADCY8 and cAMP production due to mechanical properties of the polymers affect trophic factor release (specifically VEGFB) from cells, likely through the actin pathway. The data demonstrates that through the use of the single-cell encapsulation technique the properties of NPCs can be regulated and modulated by the polymer properties.

### Experimental Section

Differentiation of Human Induced Pluripotent Stem Cell (iPSC) to NPCs

Human induced pluripotent stem cells (iPSCs) were generated from BJ fibroblasts using mRNA reprogramming factor sets leading to the overexpression of OCT4, SOX2, KLF4, and c-MYC. Culture of the human iPSC line was carried out on a matrigel-coated 6-well plate in mTeSR. Cells were incubated at 37° C. in 5% CO2, and passaged every 5-7 days with Accutase (Innovative Cell Technologies, San Diego, Calif.). iPSCs from passage 51-55 were used in these studies.

Human iPSC-derived NPCs were generated using defined conditions with minor modification to previously reported protocols.

NPC Differentiation Base Medium Formulation: DMEM/F12 (50%), Neurobasal (50%), N2-MAX (1%), B27 (1%) non-essential amino acids (NEAA) (1%), GlutaMAX (1%), 2-mercaptoethanol (0.1 mM), penicillin/streptomycin (P/S, 1% v/v) supplemented with dual SMAD inhibitors such as Dorsomorphin (1 μM) and SB431542 (1 μM).

NPC Maintenance Base Medium Formulation: DMEM/F12 (50%), Neurobasal (50%), N2-MAX (1%), B27 (1%) non-essential amino acids (NEAA) (1%), GlutaMAX (1%), 2-mercaptoethanol (0.1 mM), penicillin/streptomycin (P/S, 1% v/v) supplemented with bFGF (20 ng/mL) and EGF (20 ng/mL).

Day (0): Human iPSCs at −90% confluency were first washed with room temperature 1×DPBS without Ca₂⁺ and Mg₂⁺ once. The wash was aspirated and cells were primed by the treatment with NPC differentiation base medium for 7 d (4 mL per 6-well) under standard cell culture conditions (37° C., 5% CO2). Fresh medium was replenished every 24 hr.

Day (7): After the induction procedure, NPCs were washed with DPBS once. The cells were then detached from the plates with Accutase (1 mL per well) and incubated (37° C.). After 5 min, the side and bottom of the plate was gently rubbed to dislodge the cells from the plate surface. Then cells were collected into a 15 mL conical tube using a 10 mL serological pipette and 9 mL of DMEM/F12 containing RhoA/ROCK inhibitor, TV (2 μM), was added. Cells were centrifuged at 1,200 rpm for 5 min at room temperature. After centrifugation, the supernatant was aspirated and the cell pellet was resuspended in NPC maintenance medium+TV (2 μM). Cells were re-plated on 6-well plates previously coated with Matrigel (100,000 cells/cm²). Then, the plate with the cells was incubated under standard cell culture conditions (37° C., 5% CO2) for 24 hr.

### Single-Cell Encapsulation of NPCs Via Click Chemistry

NPCs plated on 6-well plates were maintained with NPC maintenance media. For the in vitro experiment, NPCs at 80% confluency were treated with Ac₄ManNAz (10 μM) (Kerafast, Boston, Mass.) for 2 days. The cells were washed with PBS and trypsinized from the plates with Accutase. The cells were collected by centrifugation (1,200 rpm for 5 min) and resuspended in the media containing different molecular weights of dibenzocyclooctyne-polyethyl glycol (DBCO-PEG, 5, 10, 20, and 30 kDa; 100,000 cells/mL) (BroadPharm, San Diego, Calif.) at a concentration of 1 μg/mL for 1 hr at 37° C. Subsequently, the cells were rinsed with PBS and resuspended in NPC maintenance media.

### Optimization of Single-Cell Encapsulation of NPCs

The optimal parameters including concentration of polymer and incubation time were investigated using DBCO-PEG-Cy5. After cell encapsulation with different parameters, the fluorescent intensity of the media containing cells were read by a multi-plate reader (SpectraMax, Molecular Devices, CA) (Ex: 535 nm; Em: 585 nm). In addition, the cells treated with varying concentration of DBCO-PEG-Cy5 at constant incubation time of 1 hr were imaged using fluorescent microscope (Keyence BZ-X700E, Itasca, Ill.). Controls were (1) cells treated only with Ac₄ManNAz (without polymer coating; 0 ng/mL of polymer) and (2) cells incubated with 1 μg/mL of DBCO-PEG-Cy5 without prior treatment with Ac₄ManNAz. From the optimization study, the optimal concentration of polymer (1 μg/mL) and incubation time (1 hr) were utilized for further analysis.

### Viability Assay

The viability of cells encapsulated with varying molecular weight of DBCO-PEG were evaluated using Alamar Blue assay and Live/Dead staining. For alamar blue assay, a 10% Alamar blue cell viability reagent was added to each sample and incubated at 37° C. for 3 hours in the dark. The experimental groups were cells encapsulated with different molecular weight PEG and the controls were (1) cells without encapsulation (C) (2) cells incubated with 1 μg/mL of DBCO-PEG (30 kDa) without prior treatment with Ac₄ManNAz (#), (3) cells treated with Ac₄ManNAz alone without any polymer (0 kDa) and (4) cell incubated with cell lysis buffer for 1 hour (negative control, −). After 3 hours of incubation, the absorbance of about 100 μL per sample were measured in duplicates at 570 and 600 nm using a multi-plate reader (SpectraMax, Molecular Devices, CA). The percentage reduction in absorbance (percentage viability) was calculated with respect to control-cells without encapsulation as per the manufacture protocol. For Live/Dead staining, the samples were incubated with 2 μL/mL of ethidium homodimer-1 and calcein AM for about 15 mins at 37° C. in the dark. After incubation, the cells were rinsed with 1×PBS, and imaged using a fluorescent microscope (Keyence BZ-X700).

### Transmission Electron Microscopy

The morphology of the PEG (30 kDa) coated NPCs synthesized at optimized parameters were characterized using a TEM (JEM-1400, Peabody, Mass.). Briefly, the samples were fixed with 4% paraformaldehyde in 1×PBS for 1 hour at room temperature, washed thrice in 1×PBS, re-suspended in gelatin for 5 mins and cut into blocks. The blocks were post-fixed with osmium tetroxide and uranyl acetate, serially dehydrated with ethanol, and embedded in Epon. Ultra-thin sections of the samples were sliced and examined using the JOEL-JEM 1400 TEM operated at 120 kV and the images were captured using a Gatan Onus 10.7 megapixel CCD camera. The images were processed to enhance the contrast using the Adobe Photoshop.

### AFM Force-Distance Elasticity Measurements

Force-distance (FD) measurements of cells attached to round glass cover slips coated with matrigel were performed in a liquid cell. Measurements were taken either using a Park NX-10 AFM (Park Systems, Santa Clara, Calif.) and the temperature was maintained at 37° C. throughout the experiment. Tips with a silicon oxide spherical indenter (1 μm radius, k=0.08 N/m as reported by the manufacturer, verified by a thermal tune calibration) were used on individual cells (NanoAndMore USA, Lady's Island, S.C.). Each cell was probed 2 times and a total of 20 cells was measured. Young's moduli were calculated with SPIP software (Image Metrology, Hørsholm, Denmark), which used the Hertz model for spherical indenters to fit the approach curve.

RNA Isolation and Quantitative Real-Time Polymerase Chain Reaction (qRT-PCR) Analysis

The transcription of trophic factors were measured using qRT-PCR. Total RNA was extracted from cells using a Qiagen RNeasy Plus Micro Kit (Qiagen, Germantown, Md.). After accomplishing first-strand cDNA synthesis by iScript cDNA Synthesis Kit (Bio-Rad, Hercules, Calif.), qRT-PCR was performed with TaqMan-polymerase and primers (Qiagen, Germantown, Md.) for gene expression analysis.

### ELISA Analysis

For VEGFB ELISA, the conditioned media was collected at 24 hr after single-cell encapsulation. Controls were cells without encapsulation (C). The supernatants were collected for ELISA analysis. Samples were assayed by the VEGFB Development kit from Peprotech (Peprotech, Rocky Hill, N.J.) according to the manufacturer's instructions.

cAMP measurement cAMP levels in cells were measured using the cAMP-Glo Assay (Promega, Madison, Wis.). Briefly, encapsulated-cell pellets were collected by centrifugation and treated with cAMP-Glo lysis buffer (20 μL). The lysis solution was kept with shaking at room temperature for 15 min. After the lysis process, the cAMP detection solution was added into lysis solution (40 μL) and mixed by shaking for 1 min. The solution was further incubated at room temperature for 20 min. After the incubation, Kinase-Glo reagent was added into the solution and incubated at room temperature for 10 min. The luminescence of the samples was measured with a plate-reading luminometer (SpectraMax, Molecular Devices, CA).

### Inhibition Study

After cell encapsulation with two different molecular weight polymers such as 5 kDa and 30 kDa, the cells were rinsed and resuspended in the media containing actin polymerization inhibitor (Cytochalastin D (CytoD): 2 μM) and activator (lysophosphatidic (LPA): 0.5 After the incubation for 24 hr with different pharmacological chemicals, the supernatants from different treatment groups were collected to measure VEGFB production from the cells using ELISAs as above.

### Statistical Analysis

All the data are presented as the mean±standard deviation (S.D.) of four independent experiments (biological replicates). n values indicate the number of independent experiments conducted or the number of individual experiments. An analysis of variance (ANOVA) test was used for multicomponent comparisons (n>3 independent variables) after the normal distribution was confirmed. Tukey post hoc analysis was performed to investigate the differences between variables.

### Applications

The single-cell encapsulation method presented in this invention expands polymeric encapsulation utility beyond the conventional encapsulation of a group of cells in a polymeric hydrogel depot. The technique with the ability to manipulate the cellular interaction with the extracellular environment can act as a fundamental regulator of cell function. For example, the stiffness of the polymer coating around individual cells can be modulated to induce transcriptome changes. Single-cell encapsulation of neural stem cells markedly increased the release of neurotrophic factors such as VEGF and CNTF. Moreover, stem cell differentiation is greatly influenced by its biomaterial environment. The ability to manipulate the immediate environment around each stem cell allows for accelerating the stem cell differentiation. Given the promise of stem cell therapeutics, the present invention to uniformly enhance stem cell function could prove transformative in improving efficacy and increasing feasibility by reducing the total number of cells required.

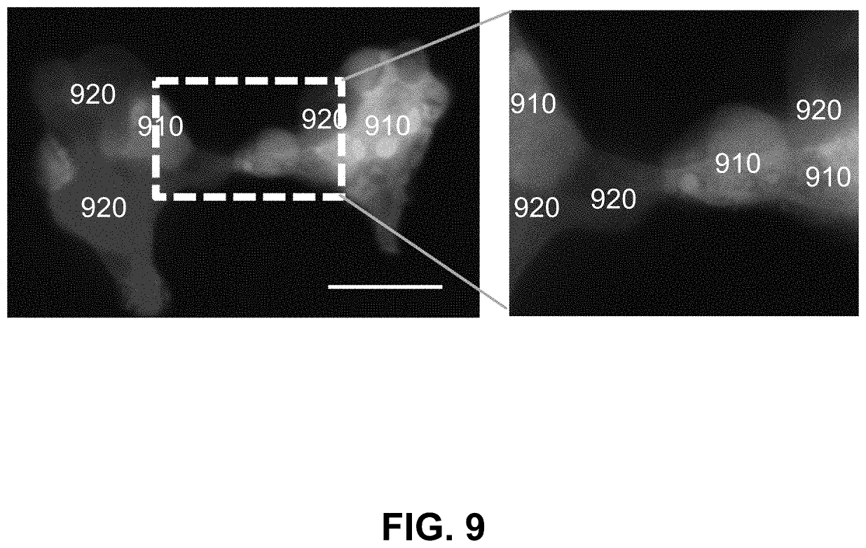

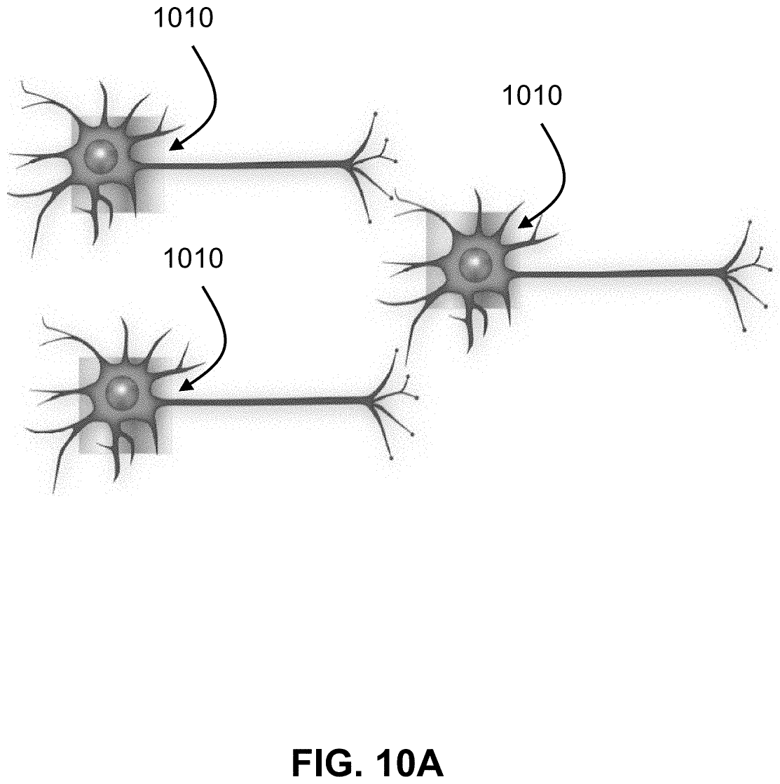

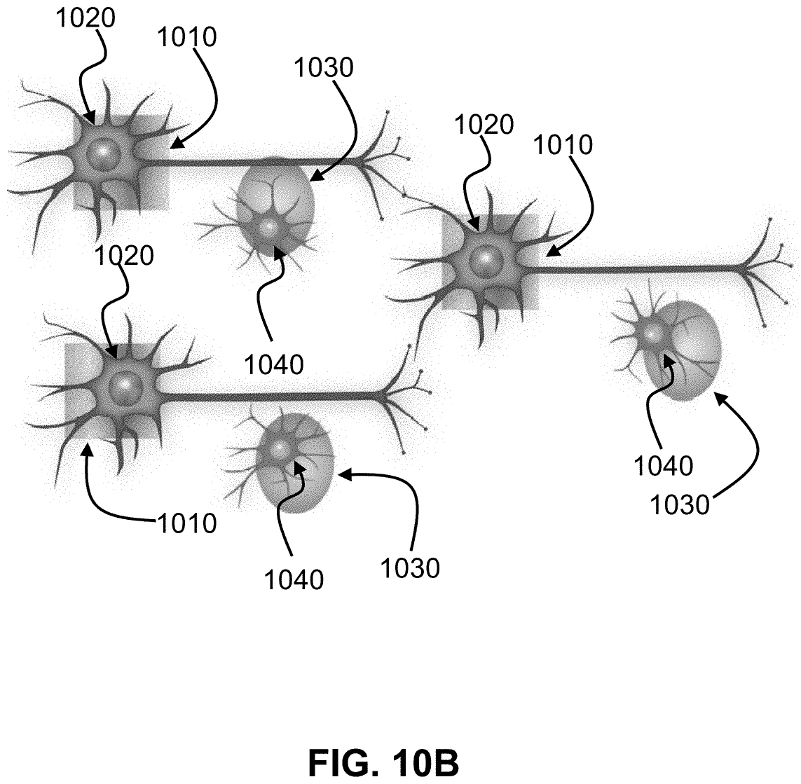

The individual cellular control achieved via the present invention expands the ability of the method to reconstruct cellular networks, in particular, neural cell networks, and in the development of organoids. Because each cell is individually coated, the polymer can be modified (1) to allow for combinations of different cell types with different polymeric coatings to form engineered combinations based in on polymeric interactions and (2) to attach moieties to each cell that could guide it to a particular binding target for single cell manipulations. For example, without limitation to the invention, one group of neural stem cells were coated with DBCO-PEG5-NH2 (green 910) and another group with DBCO-PEG5-NHS ester (red 920) (FIG. 9). The amine and NHS ester coated cells react at pH=7.5 to form an amide bond, thereby resulting in cellular attachments between the different polymer coated cells. By optimizing this platform, control of cell-to-cell interactions for organoid creation compared to traditional mixing of cells is possible. Similarly, the polymer around each cell can be modified with functional groups to preferentially react with surfaces, such as gold or silicon nitride, through chemical crosslinking (FIGS. 10A-B). Modifying the encapsulating polymer to interact with a microfabricated surface creates a novel platform to guide cells to exact locations and to better understand stem cell behavior.

1. A method of single-cell encapsulation of cells using glycoengineering and click-chemistry, comprising:
(a) treating the cells with a precursor for metabolic engineering to modify glycans in a cell membrane, wherein the treating forms reactive component A-glycans in the cell membrane suitable for a click-chemistry reaction; and
(b) suspending the treated cells in a polymer solution, wherein the polymer solution has a reactive component B suitable for the click-chemistry reaction, and wherein the reactive component A-glycans in the cell membrane react via the click-chemistry with the reaction component B thereby forming single cell polymer encapsulated cells.

2. The method as set forth in claim 1, wherein the polymer solution has polymers with different molecular weights, and wherein the cells in the formed single cell polymer encapsulated cells each have a different polymer molecular weight.

3. The method as set forth in claim 1, further comprising crosslinking the single cell polymer encapsulated cells to each other.

4. The method as set forth in claim 1, further comprising forming a network of cells or organoids by crosslinking the single cell polymer encapsulated cells to each other.

5. The method as set forth in claim 1, further comprising functionalizing the single cell polymer encapsulated cells with a reactive functionalized group.

6. The method as set forth in claim 1, further comprising attaching the single cell polymer encapsulated cells to a substrate or surface.

7. The method as set forth in claim 1, wherein the method is a copper-free method.

8. The method as set forth in claim 1, wherein the precursor is Tetraacetylated N-azidoacetyl-D-mannosamine, Tetraacetylated N-azidoacetyl-D-galactosamine, or Tetraacetylated N-azidoacetyl-D-glucosamine.

9. The method as set forth in claim 1, wherein the polymer solution is dibenzocyclooctyne-polyethyl glycol (DBCO-PEG), DBCO-PEG-NH₂, DBCO-PEG-NHSEster, DBCO-PEG-COOH, 4-arm-PEG-DBCO, or DBCO-PEG-DBCO.

10. The method as set forth in claim 1, wherein the polymer solution has polymers with different molecular weights ranging from 5 to 75 kDa.
